(19) United States
(12) Patent Application Publication
(10) Pub. No.: US 2010/0081994 A1
(43) Pub. Date: Apr. 1, 2010

(54) Self Retaining Laparoscopic Trocar System-Zisow Trocar Sleeve System

(76) Inventor: David Leslie Zisow, Baltimore, MD (US)

Correspondence Address: David Leslie Zisow, MD, 2 Marcie Woods Court, Baltimore, MD, 21208, US

(21) Appl. No.: 12/233,538

(22) Filed: Oct. 1, 2008

(51) Int. Cl. A61B 17/34

(52) U.S. Cl. 604/164.06

(57) ABSTRACT

A new concept in laparoscopic trocar systems is presented that could be used with most presently available laparoscopic trocars. The Ethicon XCEL 5 mm trocar is used as the basis for this presentation; however, any other size or system of trocars similarly configured would be compatible with the ZTSS being presented.

During laparoscopic surgery trocars often slip out of their original placement through the layers of the abdominal wall surrounding the abdomen. This causes the access to the peritoneal cavity to be lost, often at a critical moment during surgery. This necessitates various maneuvers to replace the trocar which waste time and also predispose the patient to complications such as subcutaneous emphysema and bleeding. The ZTSS would prevent this from happening.

Laparoscopic minimally invasive surgery has been rapidly developing and in the opinion of most experts, is the best way to perform complex surgery, assuming the surgeon possesses the requisite skill set for same. Entry into the abdomen (and other body sites) is generally obtained via devices known as laparoscopic trocars. These cylindrical devices have been developed in various diameters and lengths to accommodate the instrumentation to be passed through the trocar sleeve, into the body cavity, so that surgical procedures may be performed. Thus, trocars provide a route for accessing a body cavity while avoiding a large incision as required in traditional “open” surgery. Internal body parts are viewed via an endoscopic camera attached to a laparoscope with the image transmitted to a TV monitor screen. If the trocar is accidentally dislodged from its placement site, obviously, instrumentation can not be placed into the body cavity nor can camera visualization occur. Clearly, avoidance of trocar displacement is a critical aspect of successful laparoscopic surgery.

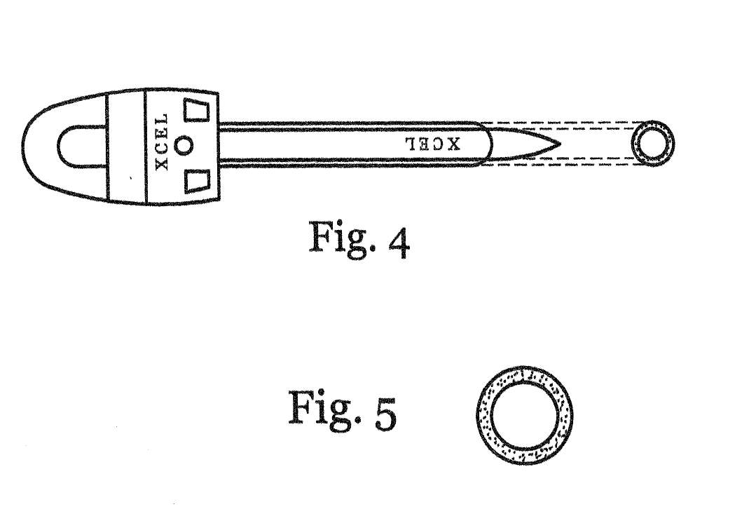

Currently available trocars are generally made of plastic and are disposable. They consist of an outer cylindrical sheath and an inner obturator with a pointed end that allows the device to be passed through the body wall layers and penetrate into the cavity to be treated. A typical trocar (Ethicon XCEL 5 mm) is seen in FIGS. 4 & 5. Once the device in penetrated into the body, the inner obturator is removed and other instrumentation may be passed through the cylindrical sheath into the body. Essentially, this trocar relies upon the frictional pressure of the body wall tissues against the cylindrical sheath to hold the trocar sheath in place. Unfortunately, this often doesn't happen, and the trocar slips out of the body cavity. This causes several significant problems. First, it slows down the operative procedure while the surgeon struggles to return the trocar to the body cavity. Second, the carbon dioxide gas used to distend the body cavity during such procedures dissects into the subcutaneous tissue spaces because the trocar is no longer preventing same. The potential for significant subcutaneous emphysema is real and can prevent the successful completion of the procedure. Lastly, trocar dislodgement often occurs at the most inopportune moment when something critical is happening. This stresses all parties involved, making what is already tedious extremely difficult. Clearly, preventing trocar dislodgement is important and with this in mind, the “Self Retaining Laparoscopic Trocar System-Zisow Trocar Sleeve System or ZTSS” has been developed and described below.

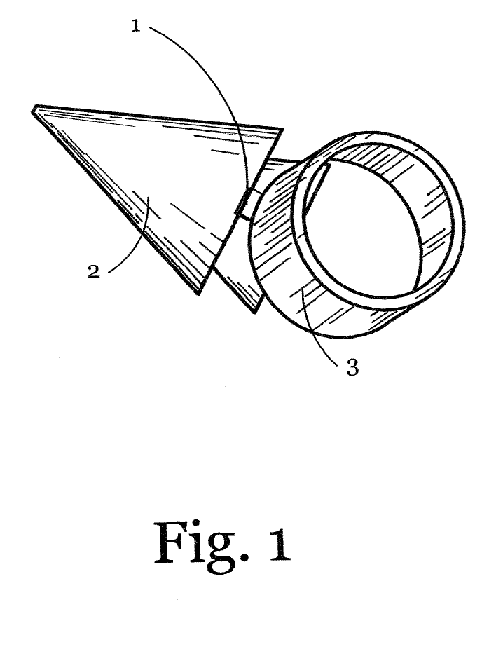

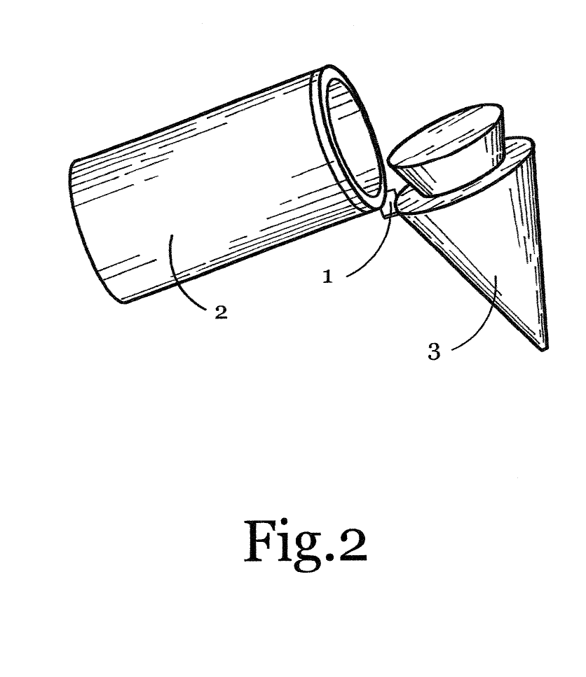

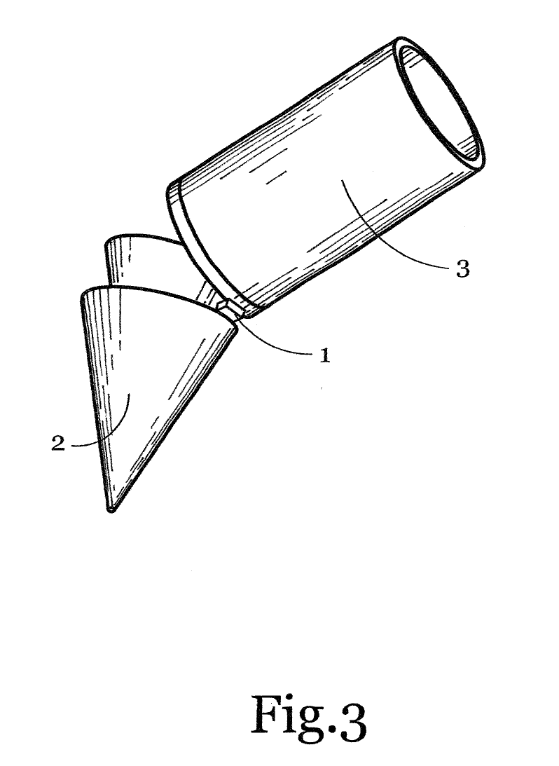

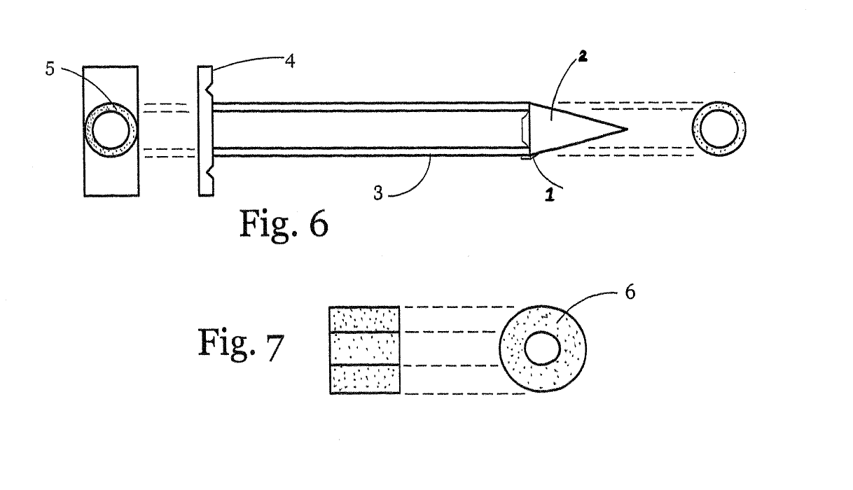

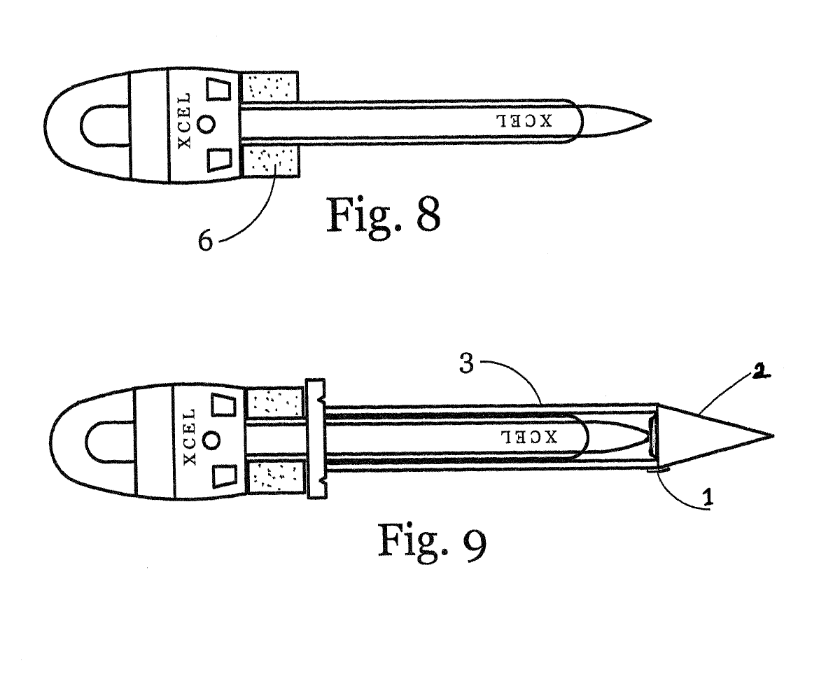

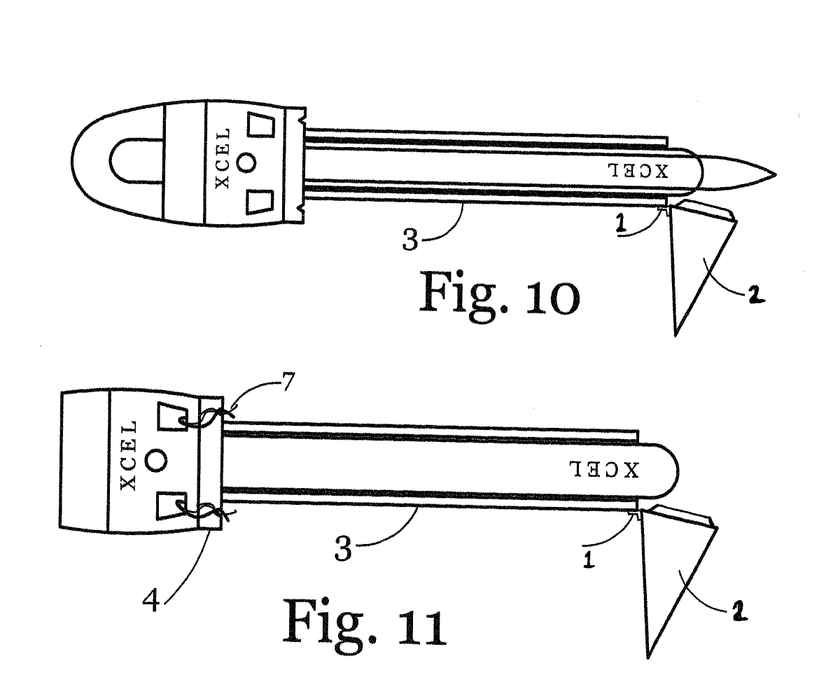

The concept behind the ZTSS is relatively simple. Instead of using a traditional two piece trocar system (outer cylindrical sheath and inner obturator for insertion purposes) the ZTSS adds a third outer sheath uniquely designed to be self retaining until the procedure is completed. FIGS. 1 through 3 are not drawn to scale. They are simply intended to demonstrate the fundamental design of the ZTSS outer sheath. Most important to notice is the fact that the tip (#2) is hinged (#1) on one side of the cylinder (#3), thus allowing the tip to be expelled from the cylindrical housing and being deployed at approximately a 90 degree angle to the cylindrical sheath. Consequently, the deployed tip prevents the cylinder from being withdrawn from the body cavity until the procedure is completed. It is the claim of this patent submission that the ZTSS will prevent unintended trocar dislodgement, thus greatly facilitating the successful performance of laparoscopic surgical procedures. FIG. 4 & 5 show the basic design of a prototypical trocar, the Ethicon XCEL 5 mm trocar. Note that the length of the device, from below the base of the handle to the tip of the obturator is 8.0 cm. FIG. 6 shows the ZTSS without the insertion governor (#6), and particularly please note the circular o-ring seal (#5) incorporated into the outer end of the ZTSS. Note the cylinder (#3) length is 6.5 cm and that the width of the solid base of the conical tip nose cone is less than that of the “Tie grooved” platform (#4). Thus, when the insertion governor (#6), in FIG. 7, is place over the XCEL trocar shaft, FIG. 8, the length from the base of the governor to the end of the obturator tip is 6.5 cm. When this unit is then placed into the ZTSS, FIG. 9, the tip of the XCEL obturator rests against the solid base of the conical tip nose cone of the base of the ZTSS. The entire unit is ready at this point for insertion into the body cavity. Once inserted into the body cavity, the XCEL unit (sheath and obturator) are carefully removed from the ZTSS leaving the nose cone (#2) of the ZTSS in the cavity. The insertion governor is then removed from the XCEL assembly and the XCEL assembly is then returned to its placement inside the ZTSS. The additional 1.5 cm of length (because the governor is no longer attached) allows the nose cone of the ZTSS to be deployed as shown in FIG. 10. The ZTSS and the XCEL are now secured together with ligature ties (#7) as shown in FIG. 11 and the obturator is removed from the XCEL. By securing the base of the XCEL to the tie grooved platform (#4) the o-ring seal (#5) creates an air tight seal at the junction between the XCEL and the ZTSS, thus preventing gas leakage at this site. Also, note that in FIG. 11 the protruding end of the XCEL cylinder continues to prevent the ZTSS nose cone from moving out of its position of 90 degree displacement. Thus, until the ties (#7) between the two devices are cut, and the XCEL cylinder is removed from the ZTSS, the nose cone of the ZTSS will prevent dislodgement of the trocar system from the peritoneal cavity.

1. The concept behind the ZTSS is relatively simple. Instead of using a traditional two piece trocar system (outer cylindrical sheath and inner obturator for insertion purposes) the ZTSS adds a third outer sheath uniquely designed to be self retaining until the procedure is completed. FIGS. 1 through 3 are not drawn to scale. They are simply intended to demonstrate the fundamental design of the ZTSS outer sheath. Most important to notice is the fact that the tip (#2) is hinged (#1) on one side of the cylinder (#3), thus allowing the tip to be expelled from the cylindrical housing (#3) and being deployed at approximately a 90 degree angle to the cylindrical sheath. Consequently, the deployed tip prevents the cylinder from being withdrawn from the body cavity until the procedure is completed. It is the claim of this patent submission that the ZTSS will prevent unintended trocar dislodgement, thus greatly facilitating the successful performance of laparoscopic surgical procedures.
